(19) United States
(12) Patent Application Publication
(10) Pub. No.: US 2024/0198127 A1
(43) Pub. Date: Jun. 20, 2024

(54) LIGHT DELIVERY DEVICE AND METHOD FOR ULTRASOUND GUIDED INTERSTITIAL PHOTODYNAMIC THERAPY

(71) Applicant: Health Research, Inc., Buffalo, NY (US)

(72) Inventors: Sergei KURENOV, West Falls, NY (US); Gal SHAFIRSTEIN, Amherst, NY (US); Nathaniel IVANICK, Clarence, NY (US); David BELLNIER, Buffalo, NY (US); Lawrence TWOREK, West Seneca, NY (US)

(21) Appl. No.: 18/557,950

(22) PCT Filed: May 2, 2022
(86) PCT No.: PCT/US2022/027354

§ 371 Date: Oct. 29, 2023

Related U.S. Application Data
(60) Provisional application No. 63/182,744, filed on Apr. 30, 2021.

(51) Int. Cl. A61N 5/06

(52) U.S. Cl. CPC A61N 5/062; A61N 2005/0626; A61N 2005/063; A61N 2005/0632; A61N 2005/0659; A61N 2005/0663

(57) ABSTRACT

A device includes a first fiber lock configured to be attached to a handle of an EUS-NA device and to selectively lock or unlock a fiber optic cable in position with respect to a sample needle. A second fiber lock is configured to be attached to abase of the EUS-NA device and to selectively lock or unlock a fiber optic cable in position. A method includes passing a fiber optic cable through a passage of the EUS-NA device such that an end of the fiber optic cable is at a tip of a sample needle of the EUS-NA device. The fiber optic cable is locked relative to the sample needle, and the sample need is advanced into the target tissue. The fiber optic cable is unlocked from the sample needle and locked relative to abase of the EUS-NA device. The sample needle is retracted such that the end of the fiber optic cable remains in the target tissue.

### CROSS-REFERENCE TO RELATED APPLICATIONS

This application claims priority to U.S. Provisional Application No. 63/182,744, filed on Apr. 30, 2021, now pending, the disclosure of which is incorporated herein by reference.

### STATEMENT REGARDING FEDERALLY SPONSORED RESEARCH

This invention was made with government support under contract no. 1R44 CA265656 awarded by the National Cancer Institute. The government has certain rights in the invention.

### BACKGROUND OF THE DISCLOSURE

Many clinical studies have shown the effectiveness of photodynamic therapy (PDT) for the treatment of solid malignancies. PDT involves the activation of a photodynamic sensitizer, retained by the tumor tissue, by visible light. Some tumors and/or lymph nodes are more readily accessible by way of body passages such as, for example, the tracheobronchial tree (e.g., bronchial tubes, etc.) or the gastrointestinal tract (e.g., esophagus, etc.). There is a long-felt need for techniques to precisely deliver light to a target tissue for PDT or other uses (for example, for characterizing the target tissue.

### BRIEF SUMMARY OF THE DISCLOSURE

The present disclosure provides a light delivery device for use with an ultrasound-guided needle aspiration device. The delivered light may be used for interstitial photodynamic therapy (I-PDT) of a cancerous tissue. The device may include or be configured for attachment to an endobronchial ultrasound (EBUS) bronchoscope with transbronchial needle (TBN), an endoscopic ultrasound fine-needle aspiration (EUS-FNA) device, or other similar devices. The device allows the precise advancement and retraction of a fiber-optic cable through a sample needle lumen, under direct ultrasound visualization. Embodiments of the device are useful for the minimally invasive treatment of locally advanced lung cancer, esophageal cancer, and/or other cancers.

### DESCRIPTION OF THE DRAWINGS

For a fuller understanding of the nature and objects of the disclosure, reference should be made to the following detailed description taken in conjunction with the accompanying drawings, in which:

FIG. 1A shows a device according to an embodiment of the present disclosure, wherein the device is shown in a needle retracted configuration;

FIG. 1B shows the device of FIG. 1A, wherein the device is shown in a needle advanced configuration

FIG. 2 shows a device according to another embodiment of the present disclosure attached to an EBUS-TBNA device;

FIG. 3 shows a frame according to an embodiment of the present disclosure;

FIG. 4 shows an example flow switch which may be used as a first fiber lock or second fiber lock;

FIG. 5 shows a portion of a device according to another embodiment of the present disclosure, wherein a portion of an attached EBUS-TBNA device is shown;

FIGS. 6A-6H show steps of a method according to another embodiment of the present disclosure, shown by illustrating manipulations during I-PDT treatment using a EBUS-TBNA device; and

FIG. 7 is a chart of a method according to another embodiment of the present disclosure.

### DETAILED DESCRIPTION OF THE DISCLOSURE

The present disclosure provides a device for precise delivery of light to a target tissue. Such a target tissue may be, for example, a tissue within, adjacent to, or making up a portion of a bodily passage such as, for example, a passage of the tracheobronchial tree (e.g., bronchial tube, etc.), a passage of the gastrointestinal tract (e.g., esophagus, etc.), or the like. In some embodiments, the device is configured for use with an endoscopic ultrasound (EUS) device or an endobronchial ultrasound (EBUS) device. An example of such a device is an EBUS for use during a bronchoscopy—a minimally-invasive procedure for visualizing the airway wall, for example, to diagnose lung cancer-causing enlarged lymph nodes. EBUS involves the insertion of a flexible bronchoscope through the mouth of an individual and into the airways of the lungs. In some cases, EUS and EBUS allow physicians to collect tissue or fluid samples from a target tissue by using a sample needle. For example, such a sample may be collected during an endoscopic ultrasound-fine needle aspiration (EUS-FNA) procedure or during the endobronchial ultrasound-guided transbronchial needle aspiration (EBUS-TBNA) procedure. Using an EBUS-TBNA, once a lesion is visualized, a sample needle is pushed through the bronchial wall into the target under direct ultrasound visualization, thereby collecting a sample by way of a lumen in the sample needle. The presently-disclosed device and methods may be configured for use with/used with EBUS-TBNA, EUS-FNA, and/or similar such techniques wherein needle aspiration/needle biopsy is utilized under an ultrasound-guided endoscopic procedure (including, for example, endobronchial procedures). For the purposes of the present application, the term “EUS-NA” will be broadly used to include EUS-FNA, EBUS-TBNA, and similar techniques. As such, where the present application recites EUS-NA, the term should be interpreted broadly to include such other similar techniques unless expressly stated otherwise.

Devices and methods of the present disclosure utilize the lumen of such a sample needle for passage of a fiber-optic cable. Such a fiber-optic cable may then be used to, for example, ablate the lesion using PDT, deliver a measurement light to the tissue, etc. In some embodiments, the sample needle may be removed (i.e., retracted) from the target tissue, while leaving the fiber-optic cable in the predefined location and position, in order to expose a diffuser of the fiber optic cable. The device allows removing a sample needle (e.g., transbronchial needle, etc.) from the target tissue while maintaining the fiber-optic cable in the predefined location in a stable position.

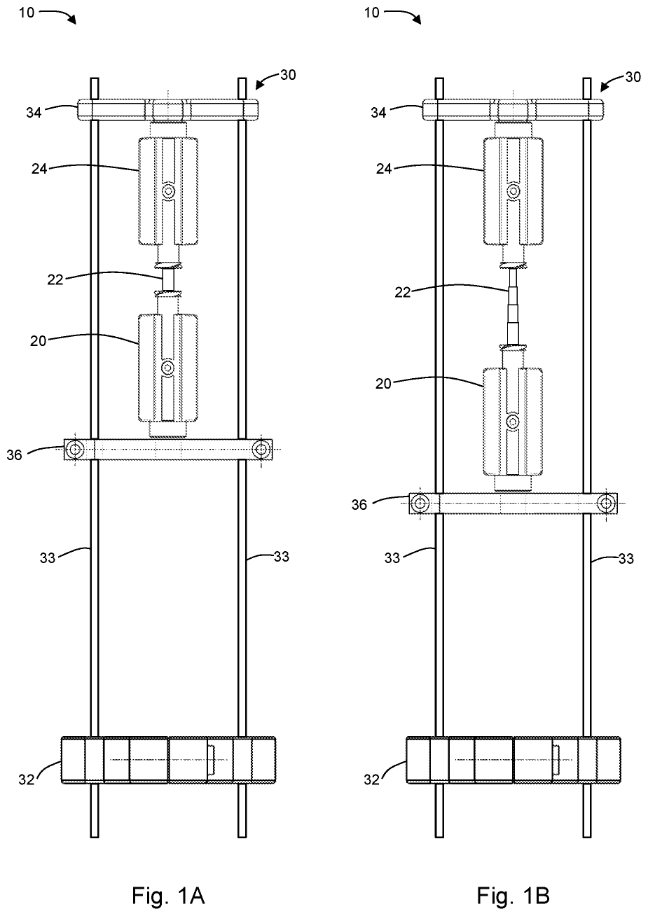

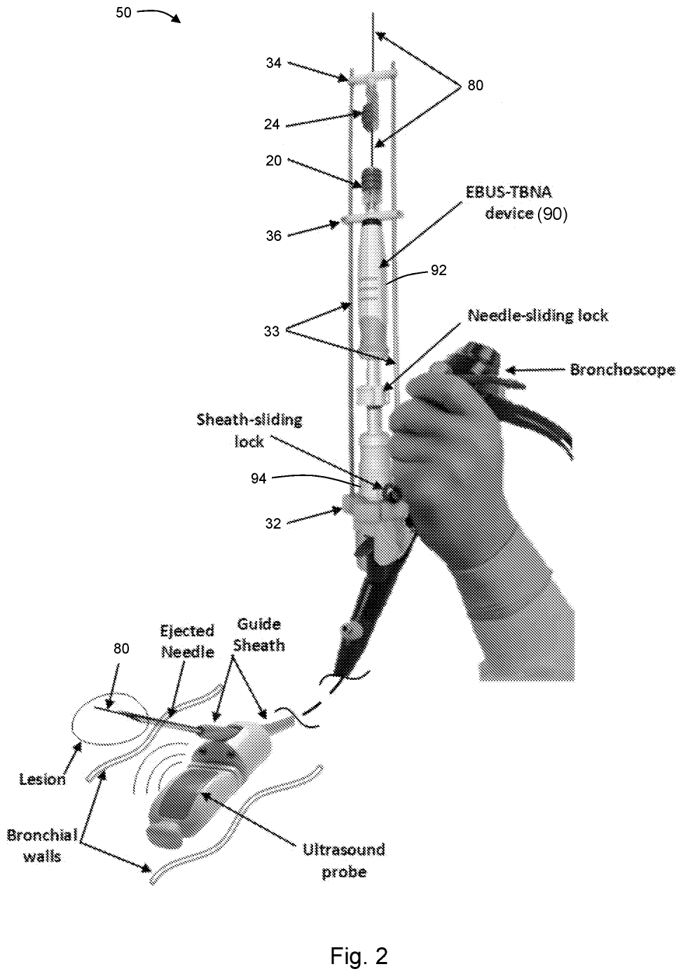

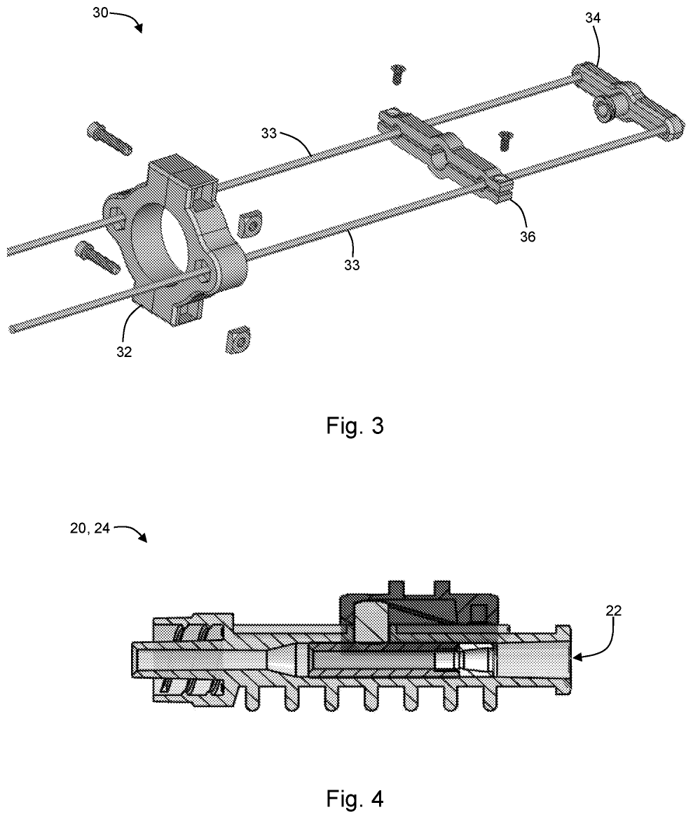

With reference to FIGS. 1A and 1i, an embodiment of the device 10 may comprise a first fiber lock 20 configured to be fixedly attached to a handle (i.e., a needle advancement handle) of an EUS-NA device, such as, for example, handle 92 of the EBUS-TBNA device 90 depicted in FIG. 2. Suitable EUS-NA devices are manufactured by, for example, Boston Scientific, Olympus, and others. The first fiber lock may include an passageway through which a fiber optic cable may pass (when such fiber optic cable is present see fiber optic cable 80 of FIG. 2). The first fiber lock is operable to selectively allow passage of the fiber optic cable through the first fiber lock when in an “unlocked” position, and stop movement of the fiber optic cable through the first fiber lock when in a “locked” position. In this way, the first fiber lock is configured to selectively lock or unlock the fiber optic cable in position with respect to a sample needle of the EUS-NA device. For example, an inline flow control switch typically used to control fluid flow through medical tubing can be used as a first fiber lock operable to selectively lock or unlock a fiber optic cable passing therethrough. An example of a suitable flow control switch 20 used as a locking device (and having passageway 22) is shown in FIG. 4. The first fiber lock may include a pad (e.g., an elastomeric material, etc.) configured to engage with the fiber optic cable to prevent damage to the fiber optic cable when locked. It should be noted that reference to “passage” of a fiber optic cable through the first fiber lock (or the second fiber lock discussed below) describes a relative movement between the fiber optic cable and the first fiber lock—e.g., the fiber optic cable may be held in place relative to the first fiber lock while the first fiber lock is moved.

The device 10 comprises a second fiber lock 24, which is configured to be fixedly attached to a base of an EUS-NA device, such as base 94 of the EBUS-TBNA device 90 depicted in FIG. 2. For example, the second fiber lock may be configured for indirect attachment to the base of the EUS-NA as further discussed below. In this way, the first fiber lock 20 and the second fiber lock 24 may move relative to one another as the handle and base of the EUS-NA device are moved relative to each other. The second fiber lock 24 may include an passageway through which the fiber optic cable 80 may pass (when such fiber optic cable present). The second fiber lock 24, is operable to selectively allow passage of the fiber optic cable when in an “unlocked” position, and stop movement of the fiber optic cable when in a “locked” position. The second fiber lock may operate in the same way as the first fiber lock or operate differently from the first fiber lock.

The first fiber lock 20 and the second fiber lock 24 form a pathway through which a fiber optic cable 80 may be fed into a lumen of the EUS-NA device. It is known that EUS-NA devices include a lumen through which a stylet may be disposed and where a sample may be extracted from the sample needle by way of, for example, a vacuum provided by a syringe. As such, the lumen passes from the body of the EUS-NA device and through the sample needle (e.g., to an orifice at/near the tip of the sample needle).

The device 10 may include a frame 30 configured to be attached to the base of the EUS-NA device. The frame 30 may include a lower bracket 32 for attachment to the base of the EUS-NA device, an upper frame 34 to which the second fiber lock 24 is attached, and one or more rods 33 connecting the lower bracket 32 to the upper bracket 34. The frame 30 may further include a middle bracket 36 configured to slide along the one or more rods 33. The middle bracket 36 may be configured to be attached to the handle of the EUS-NA device. The first fiber lock 20 may be attached to the middle bracket 36. It should be noted that as used in the present disclosure, “attachment” may be by direct attachment or indirect attachment (i.e., by way of intervening components).

In the experimental embodiment of the device 50 shown in FIG. 2, the first fiber lock 20 is shown fixedly attached to handle 92 of the EBUS-TBNA device 90 using a middle bracket 36, and the second fiber lock 24 is fixedly attached to the base 94 of the EBUS-TBNA device 90 by way of a lower bracket 32 and an upper bracket 34, where the lower bracket 32 and upper bracket 34 are connected to one another by rods 33. In the embodiment depicted in FIG. 2, the middle bracket 36 is slidingly attached to the rods 33. In this way, the first fiber lock 20 and the second fiber lock 24 are able to move relative to each other while maintaining a coaxial arrangement of the orifices through which fiber optic cable may be disposed.

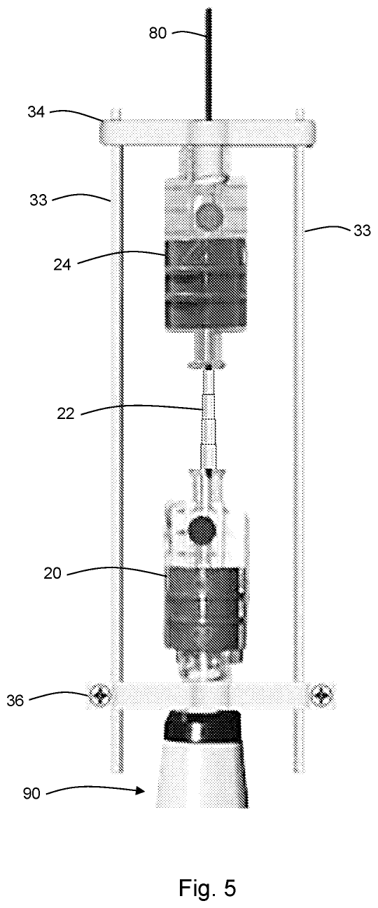

In some embodiments, the device 10 includes a fiber guide 22 to prevent the fiber optic cable from bending and/or kinking. For example, in the embodiment shown in FIGS. 1A-1B and 5, the fiber guide 22 is configured as a telescoping tube (i.e., a nesting set of tube segments) disposed between the first fiber lock and the second fiber lock a set of telescoping tubes. Other configurations for preventing bending and/or kinking of the fiber optic cable may be used.

In some embodiments, the device includes a EUS-NA device, such as, for example, an EBUS-TBNA device, and EUS-NA device, or similar device.

In some embodiments, a fiber optic cable 80 may make up a portion of a device 10. Such a fiber-optic cable may be configured to pass through the sample needle of the EUS-NA device. The sample needle may be, for example, sized as a 19 gauge, 21 gauge, 22 gauge, 25 gauge needle, or smaller or larger needle or other sizes therebetween. In some embodiments, the device may be configured for and/or include a fiber optical cable having a first optical fiber and a second optical fiber. For example, a second fiber optic cable may be provided for receiving return light—e.g., a dosimetry fiber for measuring light provided to the tissue by, for example, the first optical fiber. In some embodiments, the fiber optical cable is bifurcated so as to provide the first optical fiber and the second optical fiber. In some embodiments, a tip of the second optical fiber may be adjacent to a tip of the first optical fiber. In some embodiments, a tip of the second optical fiber may be spaced apart from a tip of the first optical fiber. For example, the tip of the first optical fiber may extend beyond the tip of the second optical fiber, or the tip of the second optical fiber may extend beyond the tip of the first optical fiber. Additional optical fibers may be provided in addition to the first optical fiber and the second optical fiber.

In some embodiments, the device 10 includes a light source in optical communication with the first optical fiber for delivery of light to the tissue. The light source may be configured for photodynamic therapy. For example, the light source may be selected to excite a photosensitizer such as, for example, Photofrin. In various examples, the light source may have a wavelength of 400 nm to 1200 nm, or 600 nm to 800 nm, or 630 nm. The light source may have a narrow wavelength range such as, for example, light provided by a laser. In some embodiments, the light source has a broad wavelength range, such as, for example, white light. In some embodiments, a light detector may be in optical communication with the second optical fiber. The light detector may be configured for, for example, dosimetry. The light detector may be, for example, a spectrometer.

The light source may be a measurement light source providing, for example, white light used to characterize the target tissue. In some embodiments, a light detector may be in optical communication with the second optical fiber. The light detector may be configured to measure irradiance (light dose rate) and/or fluence (light dose) to account for patient-specific tissue and tumor optical properties and to account for changes in fiber placements that occur after an initial pretreatment plan is generated. Similarly, the target tissue may be characterized by measuring optical properties of the tissue using light transmitted through or reflected off of the tissue. As such, light from a light source and delivered using the first optical fiber may be measured (as return light) by the light detector received by way of the second optical fiber. In some embodiments, an optical fiber (for example, the second optical fiber) may be used for dosimetry and an additional optical fiber may be used to characterize the target tissue.

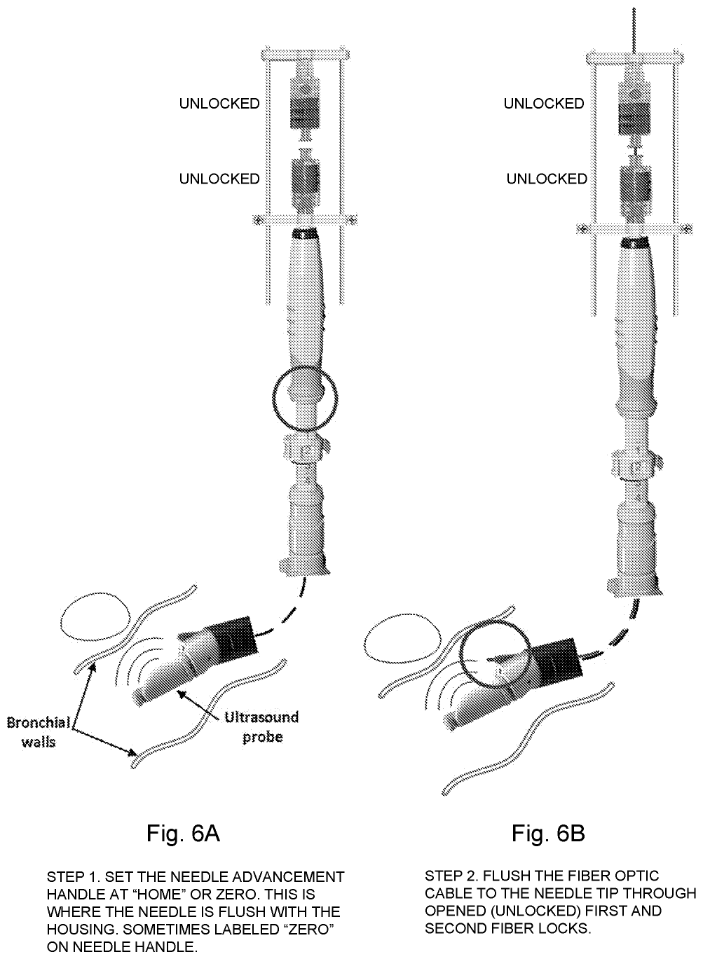

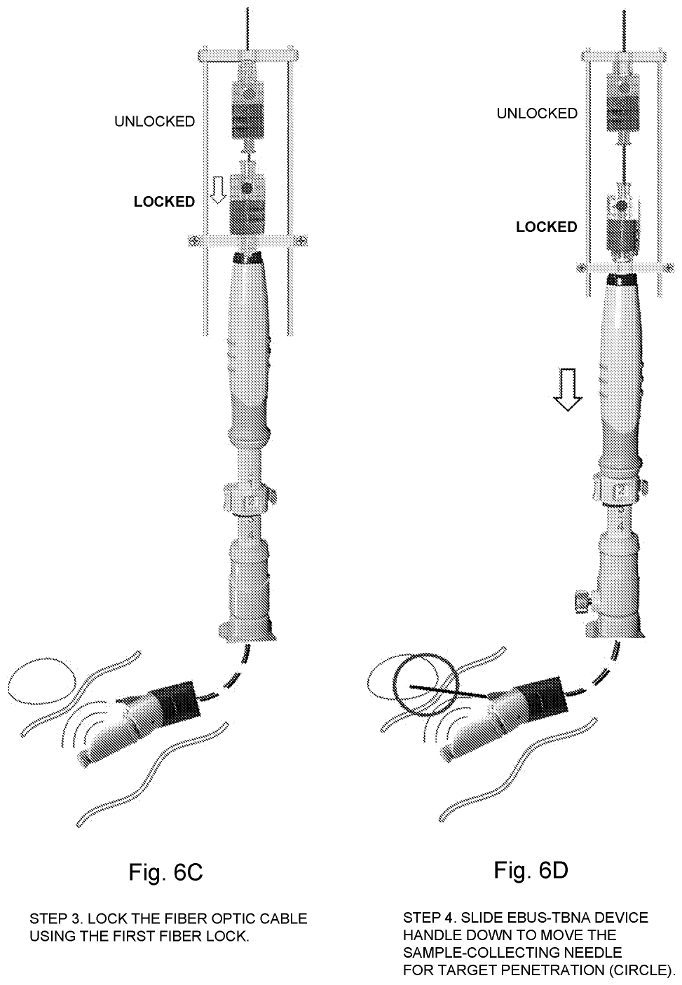

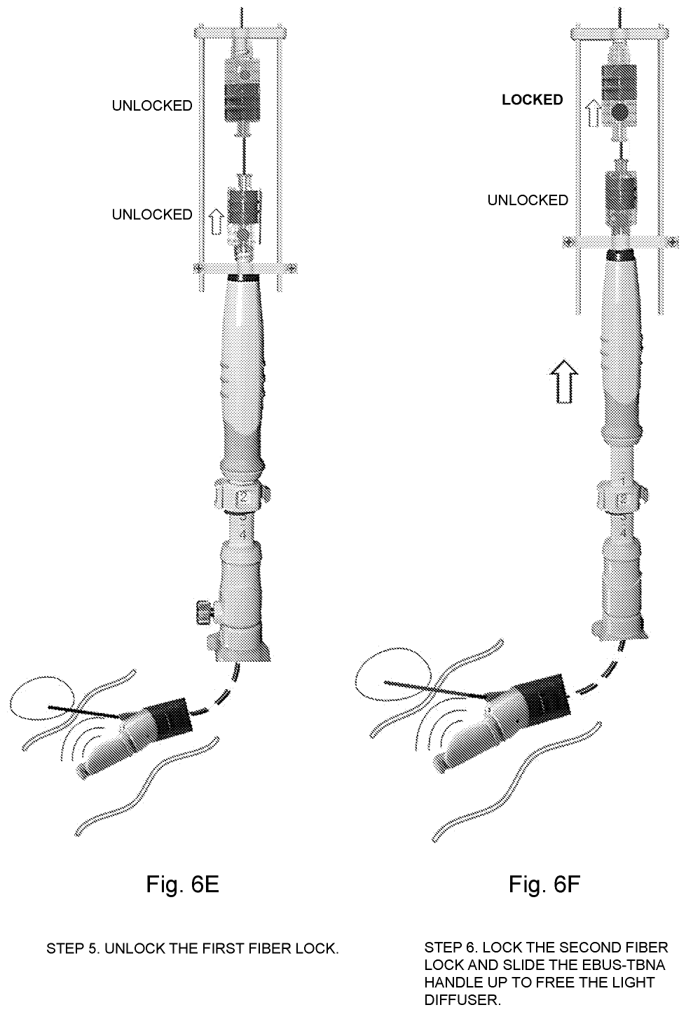

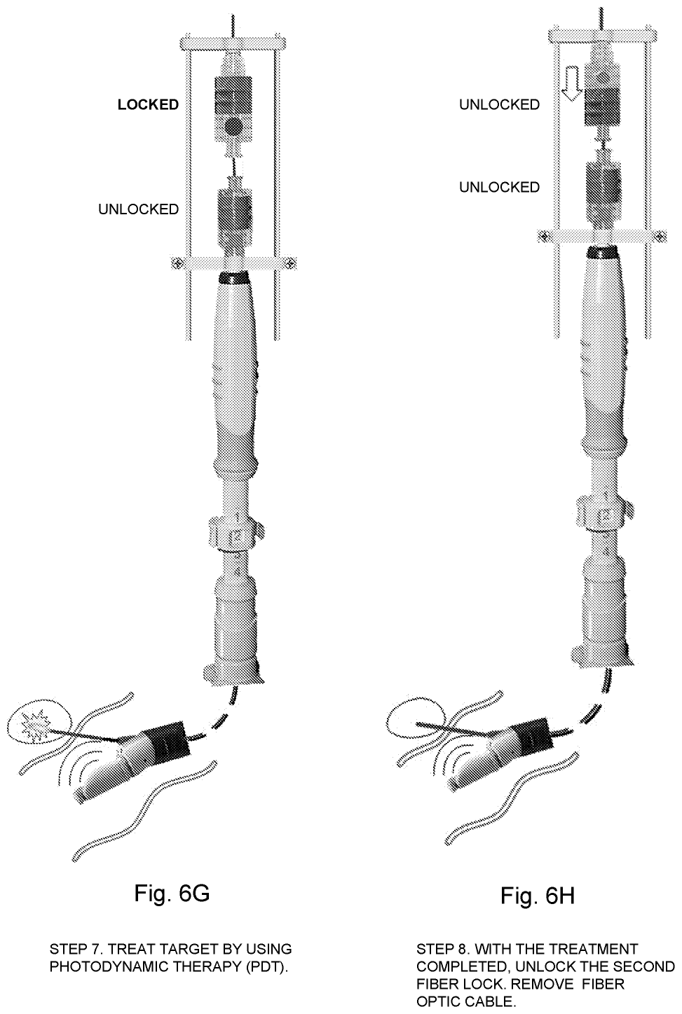

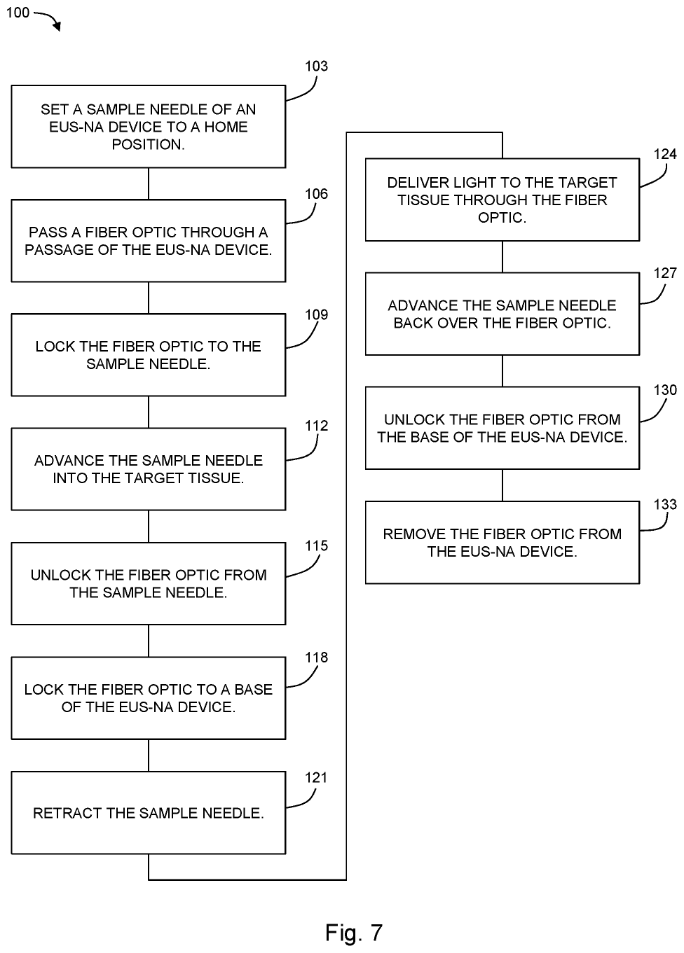

With reference to FIG. 7 (and an example embodiment using an EBUS-TBNA shown in FIGS. 6A-6H), a method 100 for using the device 10 to deliver light to a target tissue (e.g., for PDT) may include setting 103 the sample needle of an EUS-NA device is set to a “Home” position (sometimes referred to as a “Zero” position of a needle advancement handle of the EUS-NA device), where the tip of the sample needle is held at a location generally flush with a housing of an ultrasound probe (FIG. 6A). While the first fiber lock and the second fiber lock are both in the “unlocked” configuration, a fiber optic cable is passed 106 through the first fiber lock, the second fiber lock, the guide sheath of the EUS-NA, and the sample needle until an end of the fiber optic cable is at the tip of the sample needle (FIG. 6B). The ultrasound probe may be used to determine when the fiber optic cable has reached the tip of the sample needle.

The first fiber lock is locked 109 such that the fiber optic cable is fixed with respect to the first fiber lock (which is fixed to the needle advancement handle of the EUS-NA) (FIG. 6C). In this way, the fiber optic cable is fixed with respect to the sample needle such that the fiber optic cable will move with the sample needle. The sample needle is advanced 112. For example, the needle advancement handle of the EUS-NA may be moved to advance the sample needle. In this way, the sample needle (and fiber optic cable held within the sample needle) is caused to penetrate the target tissue (e.g., lesion) (FIG. 6D).

Once the sample needle and fiber optic cable have penetrated the target, the first fiber lock is unlocked 115 allowing the sample needle and fiber optic cable to move independently of each other (FIG. 6E). The second fiber lock is locked 118 such that the fiber optic cable is fixed with respect to the second fiber lock (FIG. 6F). In this way, the fiber optic cable is held in place with respect to the EUS-NA device (which is held in place relative to the target) regardless of any movement of the sample needle.

The sample needle is retracted 121 from the target. For example, the needle advancement handle may be moved to retract the sample needle from the target. Because the first fiber lock is in the unlocked position, the fiber optic cable is left to remain in its position within the target while the sample needle is retracted. Where the fiber optic cable has a diffuser at its distal end, the diffuser is no longer blocked by the sample needle and light can be applied to the target lesion by way of the fiber optic cable. The method 100 may include delivering 124 light (e.g., treatment light, measurement light, etc.) to the target tissue by way of the fiber optic cable (FIG. 6G). The delivered light may be photodynamic therapy light. For example, the delivered light may be selected to cooperate with a photosensitizer such as, for example, Photofrin. For example, the delivered photodynamic therapy light may have a wavelength of 400 nm to 1200 nm, or 600 nm to 800 nm, or 630 nm.

In some embodiments, the fiber optic cable may include a first optical fiber and a second optical fiber. Additional optical fibers may be provided in addition to the first optical fiber and the second optical fiber. In such embodiments, the method may further include delivering light to the tissue by way of the first optical fiber; and receiving a return light from the tissue by way of the second optical fiber. In some embodiments, the received return light is used for dosimetry. In some embodiments, the delivered light is a measurement light, such as, for example, a white light. The method may include characterizing the tissue using the received return light. For example, tissue having vasculature may be distinguished from connective tissue by characterizing color, attenuation, and/or other optical properties. Other target tissue optical properties may be measured for characterizing the tissue. In some embodiments, an optical fiber (for example, the second optical fiber) may be used for dosimetry and an additional optical fiber may be used to characterize the target tissue.

Once a prescribed amount of light has been applied to the target, the second fiber lock may be unlocked 127, and the fiber optic cable may be removed 130 from the target (because both of the first and second fiber locks are unlocked) (FIG. 6H). In some embodiments, the sample needle may be advanced 133 back over the fiber optic cable (for example, by moving the needle advancement handle) before the second cable is unlocked. In this way, the fiber optic cable can be maintained entirely within a needle/sheath of the EUS-NA device while the fiber optic cable is removed.

Although the present disclosure has been described with respect to one or more particular embodiments, it will be understood that other embodiments of the present disclosure may be made without departing from the spirit and scope of the present disclosure.

We claim:

1. A device for delivering light to a target tissue, comprising:
a first fiber lock configured to be attached to a handle of an EUS-NA device, and wherein the first fiber lock is configured to selectively lock or unlock a fiber optic cable in position with respect to a sample needle of the EUS-NA device;
a second fiber lock configured to be attached to a base of the EUS-NA device, and wherein the second fiber lock is configured to selectively lock or unlock a fiber optic cable in position with respect to a body of the EUS-NA device.

2. The device of claim 1, wherein the EUS-NA device is an EBUS-TBNA device or a EUS-FNA device.

3. The device of claim 1, further comprising a frame configured to be attached to the base of the EUS-NA device.

4. The device of claim 3, wherein frame comprises a lower bracket for attachment to the base of the EUS-NA device, an upper frame to which the second fiber lock is attached, and one or more rods connecting the lower bracket to the upper bracket.

5. The device of claim 4, wherein the frame further comprises a middle bracket configured to slide along the one or more rods, wherein the middle bracket is configured to be attached to the handle of the EUS-NA device, and wherein the first fiber lock is attached to the middle bracket.

6. The device of claim 1, wherein the first fiber lock and the second fiber lock are luer lock components.

7. The device of claim 1, further comprising an EUS-NA device, wherein the middle bracket is attached to a handle of the EUS-NA device, and the lower bracket is attached to a base of the EUS-NA device.

8. The device of claim 1, wherein the fiber optic cable comprises a first optical fiber and a second optical fiber.

9. The device of claim 8, wherein the first optical fiber and the second optical fiber are made from a bifurcated optical fiber.

10. The device of claim 8, further comprising a light source in optical communication with the first optical fiber for delivery of light to the tissue.

11. The device of claim 10, wherein the light source is configured for photodynamic therapy.

12. The device of claim 11, wherein the light source has a wavelength of 400 nm to 1200 nm, or 600 nm to 800 nm, or 630 nm.

13. The device of claim 10, wherein the light source is a measurement light source.

14. The device of claim 13, wherein the light source is a white light source.

15. The device of claim 10, further comprising a light detector in optical communication with the second optical fiber for measuring light received from the tissue.

16. The device of claim 15, wherein the light detector is a spectrometer.

17. A method for delivering light to a target tissue, comprising:
passing a fiber optic cable through a passage of the EUS-NA device such that an end of the fiber optic cable is at a tip of a sample needle of the EUS-NA device;
locking the fiber optic cable relative to the sample needle such that the fiber optic cable moves with the sample needle;
advancing the sample needle into the target tissue;
unlocking the fiber optic cable from the sample needle and locking the fiber optic cable relative to a base of the EUS-NA device; and
retracting the sample needle, wherein the end of the fiber optic cable remains in the target tissue.

18. The method of claim 17, wherein the EUS-NA device is an EBUS-TBNA device or an EUS-FNA device.

19. The method of claim 17, further comprising setting a sample needle of an EUS-NA device to a home position.

20. The method of claim 17, further comprising delivering photodynamic therapy light to the target tissue using the fiber optic cable.

21. The method of claim 20, wherein the delivered photodynamic therapy light has a wavelength of 400 nm to 1200 nm, or 600 nm to 800 nm, or 630 nm.

22. The method of claim 17, wherein the fiber optic cable comprises a first optical fiber and a second optical fiber.

23. The method of claim 20, further comprising:
delivering light to the tissue by way of the first optical fiber; and
receiving a return light from the tissue by way of the second optical fiber.

24. The method of claim 22, wherein the delivered light is a measurement light, and further comprising characterizing the tissue using the received return light.

25. The method of claim 23, wherein the measurement light is white light.

26. The method of claim 17, further comprising unlocking the fiber optic cable from the base and removing the fiber optic cable from the EUS-NA device.

27. The method of claim 17, further comprising:
advancing the sample needle back over the fiber optic cable;
unlocking the fiber optic cable from the base; and
removing the fiber optic cable from the EUS-NA device.
